(19) United States
(12) Patent Application Publication
(10) Pub. No.: US 2024/0152388 A1
(43) Pub. Date: May 9, 2024

(54) TASK DISPATCHING METHOD AND APPARATUS, COMPUTING AND PROCESSING DEVICE, COMPUTER PROGRAM AND COMPUTER-READABLE MEDIUM

(71) Applicant: BOE Technology Group Co., Ltd., Beijing (CN)

(72) Inventor: Pi Cao, Beijing (CN)

(73) Assignee: BOE Technology Group Co., Ltd., Beijing (CN)

(21) Appl. No.: 17/773,296

(22) PCT Filed: Jul. 27, 2021
(86) PCT No.: PCT/CN2021/108738

§ 371 Date: Apr. 29, 2022

(51) Int. Cl. G06F 9/48; G16B 30/20

(52) U.S. Cl. CPC G06F 9/485; G16B 30/20

(57) ABSTRACT

A task dispatching method and apparatus, a computing and processing device, a computer program and a computer-readable medium. The task dispatching method includes: acquiring a plurality of examination tasks to be dispatched and one or more examining apparatuses configured for processing the plurality of examination tasks, wherein the examination tasks have different task identifications; according to the examination tasks and the examining apparatus, generating a genetic sequence, the genetic sequence includes a correspondence relation between the examination tasks and the examining apparatuses processing the examination tasks, and genes in the genetic sequence are the task identifications of the examination tasks; and performing iterative updating to the genetic sequence, till an iteration stopping condition is satisfied, and according to the genetic sequence that has been updated, dispatching the examination tasks.

### TECHNICAL FIELD

The present disclosure relates to the technical field of computers, and particularly relates to a task dispatching method and an apparatus, a computing and processing device, a computer program and a computer-readable medium.

### BACKGROUND

According to the Statistical Bulletin of the Development of the Undertaking of Hygiene and Health of China in 2019, the total quantity of diagnosed or treated people in the medical and sanitary institutions all over the country reached 8.72 billion (with a growth rate of 4.9%), and the time quantity per capita of diagnosis or treatment reaches 6.2 times, which has already exceed the globally average level of 5.42 times. The capacity of deploying large-scale medical facilities directly influences the capacity for diagnosis and treatment of medical and sanitary institutions.

### SUMMARY

The present disclosure provides a task dispatching method, wherein the task dispatching method includes:
acquiring a plurality of examination tasks to be dispatched and one or more examining apparatuses configured for processing the plurality of examination tasks, wherein the examination tasks have different task identifications;
according to the examination tasks and the examining apparatus, generating a genetic sequence, the genetic sequence includes a correspondence relation between the examination tasks and the examining apparatuses processing the examination tasks, and genes in the genetic sequence are the task identifications of the examination tasks; and
performing iterative updating to the genetic sequence, till an iteration stopping condition is satisfied, and according to the genetic sequence that has been updated, dispatching the examination tasks;
wherein the step of the iterative updating includes:
determining an apparatus operation duration and a task-switching parameter that correspond to the genetic sequence before the updating, wherein the apparatus operation duration refers to a maximum value of operation durations during which the one or more examining apparatuses completely process the plurality of examination tasks according to the genetic sequence before the updating, the task-switching parameter is for characterizing a duration that is required by a first examining apparatus corresponding to the maximum value for switching and processing a first examination task, and the first examination task refers to an examination task in the genetic sequence before the updating that has a correspondence relation with the first examining apparatus;
according to the apparatus operation duration and the task-switching parameter, determining an adaptive value of the genetic sequence before the updating, wherein each of the apparatus operation duration and the task-switching parameter has a relation of negative correlation with the adaptive value; and
according to the adaptive value, selecting a high-quality genetic sequence from a plurality of genetic sequences before the updating, and generating the genetic sequence that has been updated based on the high-quality genetic sequence.

In an optional embodiment, the step of determining the apparatus operation duration that corresponds to the genetic sequence before the updating includes:
acquiring a first historical operation duration of a second examining apparatus, wherein the second examining apparatus refers to any one of the one or more examining apparatuses, and the first historical operation duration refers to a duration during which the second examining apparatus has already started operating before the examination tasks are processed;
according to examination durations of predetermined examination types, and an examination type of a second examination task, determining an examination duration of the second examination task, wherein the second examination task refers to an examination task in the genetic sequence before the updating that has a correspondence relation with the second examining apparatus;
summing the first historical operation duration with the examination durations of the second examination tasks, to obtain a pre-estimated operation duration of the second examining apparatus; and
determining a maximum value of the pre-estimated operation durations of the one or more examining apparatuses to be the apparatus operation duration.

In an optional embodiment, the step of determining the task-switching parameter that corresponds to the genetic sequence before the updating includes:
according to preset similarities between any two examination types, and examination types of the first examination tasks, determining first similarities between any two successive instances of the first examination tasks in the genetic sequence before the updating;
averaging the first similarities, to obtain a similarity parameter quantity; and
calculating a reciprocal of the similarity parameter quantity, to obtain the task-switching parameter.

In an optional embodiment, the step of, according to the apparatus operation duration and the task-switching parameter, determining the adaptive value of the genetic sequence before the updating includes:
acquiring a reference operation duration; and
calculating a difference between the reference operation duration and the apparatus operation duration, and performing weighted summation to the difference and a reciprocal of the task-switching parameter, to obtain the adaptive value.

In an optional embodiment, the step of acquiring the reference operation duration includes:
acquiring a second historical operation duration of a third examining apparatus, wherein the third examining apparatus refers to any one of the one or more examining apparatuses, and the second historical operation duration refers to a duration during which the third examining apparatus has already started operating before the examination tasks are processed;
according to examination durations of predetermined examination types, the examination types of the examination tasks and an order of acquiring the examination tasks, determining a third examination task required to be processed by the third examining apparatus and an examination duration of the third examination task;
summing the examination durations of the third examination tasks and the second historical operation duration, to obtain a sequential operation duration of the third examining apparatus; and
determining a maximum value of the sequential operation durations of the one or more examining apparatuses to be the reference operation duration.

In an optional embodiment, a set formed by all of the genetic sequences before the updating is a population, and the step of, according to the adaptive value, selecting the high-quality genetic sequence from the plurality of genetic sequences before the updating includes:
calculating a sum of adaptive values of all of the genetic sequences in the population, to obtain a population adaptive value;
calculating a ratio of an adaptive value of a first genetic sequence to the population adaptive value, wherein the first genetic sequence refers to any one of all of the genetic sequences before the updating; and
by using a roulette wheel selection method, by using the ratio as a probability of being-selected, selecting a genetic sequence from the population, wherein the selected genetic sequence is the high-quality genetic sequence.

In an optional embodiment, before the step of calculating the sum of adaptive values of all of the genetic sequences in the population, the method further includes:
if an adaptive value of a second genetic sequence is less than a first preset threshold, deleting the second genetic sequence from the population; and
if the adaptive value of the second genetic sequence is equal to the first preset threshold, adjusting the adaptive value of the second genetic sequence to be a sum of the first preset threshold and a specified numerical value;
wherein the second genetic sequence refers to any one of all of the genetic sequences before the updating;
the first preset threshold is greater than or equal to 0; and
the specified numerical value is greater than 0 and less than or equal to 0.1.

In an optional embodiment, the step of generating the genetic sequence that has been updated based on the high-quality genetic sequence includes at least one of the following steps:
according to a first probability, replicating the high-quality genetic sequence, to generate the genetic sequence that has been updated;
according to a second probability, performing cross treatment to two different instances of the high-quality genetic sequence, to generate the genetic sequence that has been updated, and
according to a third probability, performing variation treatment to genetic sequences obtained after cross treatment, to generate the genetic sequence that has been updated.

In an optional embodiment, the step of, according to the examination tasks and the examining apparatus, generating the genetic sequence includes:
allocating the plurality of examination tasks randomly to the one or more examining apparatuses, and according to a correspondence relation between the allocated examination tasks and the examining apparatuses, determining the genetic sequence.

In an optional embodiment, the genetic sequence that has been updated is a plurality of genetic sequences that have been updated, and the step of, according to the genetic sequence that has been updated, dispatching the examination tasks includes:
calculating adaptive value of the genetic sequences that have been updated;
selecting a genetic sequence whose adaptive value is greater than or equal to a second preset threshold as a target genetic sequence from the plurality of genetic sequences that have been updated; and
according to the target genetic sequence, dispatching the examination tasks.

In an optional embodiment, the task identifications are identifications that are determined according to the order of acquiring the examination tasks, and before the step of determining the apparatus operation duration and the task-switching parameter that correspond to the genetic sequence before the updating, the method further includes:
according to an examination order of the examination tasks in a third genetic sequence, determining examination identifications of the examination tasks, wherein the third genetic sequence refers to any one of all of the genetic sequences before the updating;
calculating differences between the examination identifications and the task identifications; and
if any one of the differences is less than a third preset threshold, deleting the third genetic sequence.

The present disclosure provides a task dispatching apparatus, wherein the task dispatching apparatus includes:
an acquiring module configured for acquiring a plurality of examination tasks to be dispatched and one or more examining apparatuses configured for processing the plurality of examination tasks, wherein the examination tasks have different task identifications;
a generating module configured for, according to the examination tasks and the examining apparatus, generating a genetic sequence, the genetic sequence includes a correspondence relation between the examination tasks and the examining apparatuses processing the examination tasks, and genes in the genetic sequence are the task identifications of the examination tasks; and
an updating module configured for performing iterative updating to the genetic sequence, till an iteration stopping condition is satisfied, and according to the genetic sequence that has been updated, dispatching the examination tasks;
wherein the updating module is particularly configured for:
determining an apparatus operation duration and a task-switching parameter that correspond to the genetic sequence before the updating, wherein the apparatus operation duration refers to a maximum value of operation durations during which the one or more examining apparatuses completely process the plurality of examination tasks according to the genetic sequence before the updating, the task-switching parameter is for characterizing a duration that is required by a first examining apparatus corresponding to the maximum value for switching and processing a first examination task, and the first examination task refers to an examination task in the genetic sequence before the updating that has a correspondence relation with the first examining apparatus;
according to the apparatus operation duration and the task-switching parameter, determining an adaptive value of the genetic sequence before the updating, wherein each of the apparatus operation duration and the task-switching parameter has a relation of negative correlation with the adaptive value; and
according to the adaptive value, selecting a high-quality genetic sequence from a plurality of genetic sequences before the updating, and generating the genetic sequence that has been updated based on the high-quality genetic sequence.

The present disclosure provides a computing and processing device, wherein the computing and processing device includes:
a memory storing a computer-readable code; and
one or more processors, wherein when the computer-readable code is executed by the one or more processors, the computing and processing device implements the task dispatching method according to any one of the embodiments.

The present disclosure provides a computer program, wherein the computer program includes a computer-readable code, and when the computer-readable code is executed in a computing and processing device, the computer-readable code causes the computing and processing device to implement the task dispatching method according to any one of the embodiments.

The present disclosure provides a computer-readable medium, wherein the computer-readable medium stores the task dispatching method according to any one of the embodiments.

The above description is merely a summary of the technical solutions of the present disclosure. In order to more clearly know the elements of the present disclosure to enable the implementation according to the contents of the description, and in order to make the above and other purposes, features and advantages of the present disclosure more apparent and understandable, the particular embodiments of the present disclosure are provided below.

### BRIEF DESCRIPTION OF THE DRAWINGS

In order to more clearly illustrate the technical solutions of the embodiments of the present disclosure or the related art, the figures that are required to describe the embodiments or the related art will be briefly introduced below. Apparently, the figures that are described below are embodiments of the present disclosure, and a person skilled in the art can obtain other figures according to these figures without paying creative work. It should be noted that the proportions in the drawings are merely illustrative and do not indicate the actual proportions.

FIG. 1 schematically shows a flow chart of a task dispatching method;

FIG. 2 schematically shows a block diagram of a task dispatching apparatus;

FIG. 3 schematically shows a block diagram of a computing and processing device for executing the method according to the present disclosure; and

FIG. 4 schematically shows a storage unit for maintaining or carrying a program code for executing the method according to the present disclosure.

### DETAILED DESCRIPTION

In order to make the objects, the technical solutions and the advantages of the embodiments of the present disclosure clearer, the technical solutions of the embodiments of the present disclosure will be clearly and completely described below with reference to the drawings of the embodiments of the present disclosure. Apparently, the described embodiments are merely certain embodiments of the present disclosure, rather than all of the embodiments. All of the other embodiments that a person skilled in the art obtains on the basis of the embodiments of the present disclosure without paying creative work fall within the protection scope of the present disclosure.

Currently, hospitals commonly allocate the examining apparatuses to patients in the mode of “First Come First Serve”. This allocating mode results in cases in which some of the examining apparatuses have a too long operation duration, while the other apparatuses are idle. Furthermore, the allocating mode of First Come First Serve does not take into consideration the examination types of two successive patients that use the same one examining apparatus, and if the examination types of two successive patients that use the same one examining apparatus are different, the examining apparatus might require to be adjusted and switched, which results in unnecessary waste in time.

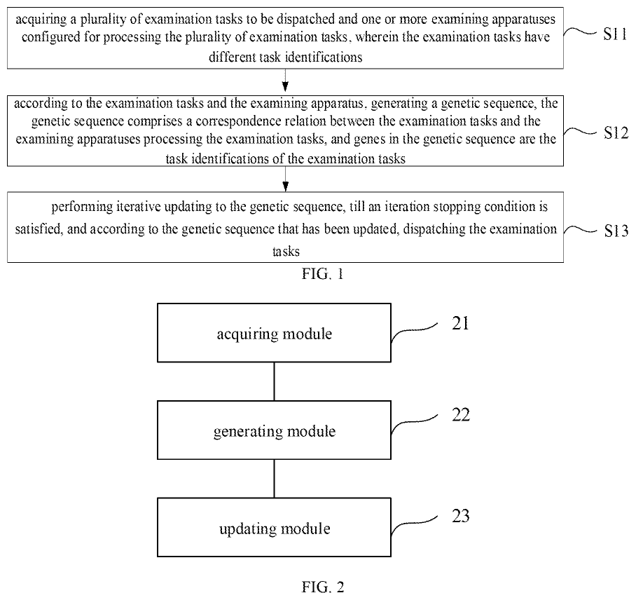

FIG. 1 is a flow chart of the task dispatching method according to an illustrative embodiment. As shown in FIG. 1, the method may include the following steps.

Step S11: acquiring a plurality of examination tasks to be dispatched and one or more examining apparatuses configured for processing the plurality of examination tasks, wherein the examination tasks have different task identifications.

The executive subject of the present embodiment may be a computer device, wherein the computer device has a task dispatching apparatus, and executes the task dispatching method according to the present embodiment by using the task dispatching apparatus. The computer device may, for example, be a smartphone, a tablet personal computer, a personal computer and so on, which is not limited in the present embodiment.

The executive subject of the present embodiment may acquire the examination tasks in various manners. For example, the executive subject may be connected to a scanning device, and acquire the examination tasks collected by the scanning device. The scanning device may collect the examination tasks by scanning the bar code of a health insurance card or inspection sheet of patients.

The task identifications of the examination tasks may, for example, be determined according to the order of acquiring the examination tasks; in other words, the task identifications may be identifications that are determined according to the time of the scanning of the examination tasks. The mode of determining the task identifications is not limited in the present embodiment.

The executive subject of the present embodiment may acquire the examining apparatuses configured for processing the examination tasks in various manners. For example, the executive subject may acquire the information of the examining apparatuses configured for processing the examination tasks that is pre-provided in a managed account. The executive subject may also be connected to the examining apparatuses, and acquire the information of the examining apparatuses via connecting interfaces.

The examining apparatus may, for example, be a Magnetic Resonance Imaging (MRI) apparatus, and the examination tasks that it may process include examination types such as head magnetic resonance imaging, thoracic-vertebrae magnetic resonance imaging and abdomen magnetic resonance imaging and other examination types, which is not limited in the present embodiment.

The examination types of the examination tasks may be the same or different, which is not limited in the present embodiment.

In the present embodiment, both of the examination tasks and the examining apparatuses are limited. It is assumed that, at any one moment, one examining apparatus can merely process one examination task, and the examining apparatus is not interrupted during the process of processing the examination task.

Assuming that, currently, the quantity of the patients waiting for the examination in an examination room is n (n≥2), and each of the patients merely performs one examination item, then the quantity of the examination tasks to be dispatched is n, wherein the task identifications of the n examination tasks are 1, 2, . . . , n. The n examination tasks form a set J={H, A, H, T, . . . , H}, and the set J includes n examination tasks, which involve three examination types H, A and T, wherein H represents head magnetic resonance imaging, t represents thoracic-vertebrae magnetic resonance imaging, and A represents abdomen magnetic resonance imaging.

It is assumed that the quantity of the examining apparatuses is m (m≥1), whose apparatus identifications are 1, 2, . . . , m.

Step S12: according to the examination tasks and the examining apparatus, generating a genetic sequence, the genetic sequence includes a correspondence relation between the examination tasks and the examining apparatuses processing the examination tasks, and genes in the genetic sequence are the task identifications of the examination tasks.

Wherein the genetic sequence refers to a sequence obtained by arranging the task identifications of the plurality of examination tasks in a certain order, and the order of arrangement of the examination tasks in the genetic sequence is the examination order. In the genetic sequence, the correspondence relation between the examination tasks and the examining apparatuses may be expressed by Bᵢₘ, wherein Bᵢₘ indicates that the i^{th} examination task (the examination task whose task identification is i) is processed by the m^{th} examining apparatus (the examining apparatus whose apparatus identification is m).

In the present embodiment, one genetic sequence corresponds to one dispatching theme, and corresponds to one chromosome or one individual in a genetic algorithm.

In a particular embodiment, the step of, according to the examination tasks and the examining apparatuses, generating the genetic sequence has various implementations. In an optional embodiment, and this step may include allocating the plurality of examination tasks randomly to the one or more examining apparatuses, and according to a correspondence relation between the allocated examination tasks and the examining apparatuses, generating a genetic sequence.

In the present embodiment, by generating the genetic sequence randomly, the initialization of the genetic sequence is completed before the iterative updating.

Step S13: performing iterative updating to the genetic sequence, till an iteration stopping condition is satisfied, and according to the genetic sequence that has been updated, dispatching the examination tasks.

In the present embodiment, the iteration stopping condition may have various implementations.

For example, in an implementation, the iteration stopping condition is that the iteration time quantity reaches a preset time quantity.

After the iteration stopping condition has been satisfied, if the genetic sequence that has been updated is a plurality of genetic sequences that have been updated, then one of the genetic sequences is selected as the target genetic sequence, and the examination tasks are dispatched according to the target genetic sequence. The mode of selecting the target genetic sequence will be described in the subsequent embodiments. If the quantity of the genetic sequence that has been updated is one, then the genetic sequence that has been updated is used as the target genetic sequence. In the present implementation, the preset time quantity may be preset by a person skilled in the art empirically, and is not limited in the embodiments of the present disclosure.

In another implementation, the iteration stopping condition is that the quantity of the genetic sequence that has been updated is one.

Particularly, if, after a certain time of iteration process has been completed, the quantity of the obtained genetic sequence that has been updated is one, then the genetic sequence that has been updated may be used as the target genetic sequence, and the examination tasks are dispatched according to the target genetic sequence.

The present embodiment may use any one of the above modes as the iteration stopping condition, and, certainly, may also use other iteration stopping conditions, and the iteration stopping condition is not particularly limited in the present embodiment.

In the step S13, the step of the iterative updating includes:
firstly, determining an apparatus operation duration and a task-switching parameter that correspond to the genetic sequence before the updating, wherein the apparatus operation duration refers to a maximum value of operation durations during which the one or more examining apparatuses completely process the plurality of examination tasks according to the genetic sequence before the updating, the task-switching parameter is for characterizing a duration that is required by a first examining apparatus corresponding to the maximum value for switching and processing a first examination task, and the first examination task refers to an examination task in the genetic sequence before the updating that has a correspondence relation with the first examining apparatus, wherein the examining apparatus corresponding to the maximum value is the first examining apparatus;
subsequently, according to the apparatus operation duration and the task-switching parameter, determining an adaptive value of the genetic sequence before the updating, wherein each of the apparatus operation duration and the task-switching parameter has a relation of negative correlation with the adaptive value, wherein the adaptive value is used to characterize the capacity of the genetic sequence before the updating of processing the examination tasks in the task dispatching; and
subsequently, according to the adaptive value, selecting a high-quality genetic sequence from a plurality of genetic sequences before the updating, and generating the genetic sequence that has been updated based on the high-quality genetic sequence.

In the present embodiment, the apparatus operation duration refers to the maximum value of the operation durations during which the one or more examining apparatuses completely process the plurality of examination tasks. After the plurality of examination tasks have been completely processed, regarding each of the examining apparatuses, the operation duration of the examining apparatus may include the duration that is required by completely processing the examination task corresponding to the examining apparatus in the genetic sequence before the updating, and may further include the duration during which the examining apparatus has already started operating before processing those examination tasks. The particular mode of determining the apparatus operation duration will be described in detail in the subsequent embodiments.

The apparatus operation duration is the maximum value of the operation durations of the one or more examining apparatuses. The examining apparatus corresponding to the maximum value or the apparatus operation duration is the first examining apparatus, the examination task in the genetic sequence before the updating that has a correspondence relation with the first examining apparatus is the first examination task, and the first examination task is processed by the first examining apparatus.

The task-switching parameter may characterize the duration that is required by switching the first examination tasks by the first examining apparatus in the process of processing all of the first examination tasks. When the examination types of two successive first examination tasks are the same or similar, the switching operation of the first examining apparatus can be reduced, to realize quick locating, whereby reducing the switching duration. Therefore, the task-switching parameter may be determined according to the similarity between any two successive first examination tasks, and such a mode of determination will be described in detail in the subsequent embodiments.

Moreover, the task-switching parameter may also be determined by, according to preset durations of switching between any two examination types and the examination types of the first examination tasks, determining the duration of switching between any two successive first examination tasks in the genetic sequence before the updating, and summing the switching durations.

The mode of determining the task-switching parameter is not particularly limited in the present embodiment.

In a genetic algorithm, the genetic sequence having a higher adaptive value has a higher probability of being selected. Therefore, in order to minimize the apparatus operation duration, the apparatus operation duration and the adaptive value have a relation therebetween of negative correlation. In other words, regarding each of the genetic sequences before the updating, the apparatus operation duration of that genetic sequence and the probability of being selected of the genetic sequence have a relation of negative correlation.

Because the apparatus operation duration of that genetic sequence and the probability of the genetic sequence being selected have a relation of negative correlation, in the process of each time of the iterative updating, the genetic sequence having a lower apparatus operation duration has a higher probability of being selected. Accordingly, by performing multiple times of iterative updating to the genetic sequences, the examination order of the examination tasks can be optimized and adjusted, thereby reaching the objective of shortening the apparatus operation duration (i.e., the longest examination duration of the apparatuses).

In order to further increase the utilization ratio of the apparatuses, minimize the task-switching parameter, and shorten the switching duration occupied by the switching of the examination tasks of the same one examining apparatus, the task-switching parameter and the adaptive value have a relation therebetween of negative correlation. In other words, regarding each of the genetic sequences before the updating, the task-switching parameter of that genetic sequence and the probability of being selected of the genetic sequence have a relation of negative correlation.

Because the task-switching parameter of that genetic sequence and the probability of being selected of the genetic sequence have a relation of negative correlation, in the process of each time of the iterative updating, the genetic sequence having a lower task-switching parameter has a higher probability of being selected. Accordingly, by performing multiple times of iterative updating to the genetic sequences, the examination order of the examination tasks can be optimized and adjusted, thereby reaching the objective of shortening the duration of task switching.

In the present embodiment, an adaptive value function may be established with the object of minimizing the apparatus operation duration and the task-switching parameter. In a particular implementation, the particular function relations of the adaptive value with the apparatus operation duration and the task-switching parameter may be adjusted according to actual situations, and are not limited in the present embodiment. The particular mode of determining the adaptive value will be described in detail in the subsequent embodiments.

In a particular implementation, the step of selecting the high-quality genetic sequence from the plurality of genetic sequences before the updating according to the adaptive value may be performed in various manners. For example, the step may include selecting the genetic sequences by using the proportion of the adaptive values of the genetic sequences before the updating in the population adaptive value (the population adaptive value refers to the sum of the adaptive values of all of the genetic sequences before the updating) as the probability of being-selected. The step may also include sorting the adaptive values of the genetic sequences before the updating, and selecting according to the positions in the sorting, and so on. The former implementation will be described in detail in the subsequent embodiments.

In the present embodiment, the step of generating the genetic sequence that has been updated based on the high-quality genetic sequence may be performed in various manners. For example, the high-quality genetic sequence may undergo at least one of replication, cross treatment and variation treatment, to obtain the genetic sequence that has been updated.

In a particular implementation, the step of generating the genetic sequence that has been updated based on the high-quality genetic sequence may include at least one of the following steps: according to a first probability, replicating the high-quality genetic sequence, to generate the genetic sequence that has been updated; according to a second probability, performing cross treatment to two different instances of the high-quality genetic sequence, to generate the genetic sequence that has been updated; and according to a third probability, performing variation treatment to genetic sequences obtained after cross treatment, to generate the genetic sequence that has been updated.

In the present implementation, by performing the cross treatment to the two different high-quality genetic sequences, the obtaining of a locally optimum genetic sequence can be prevented, which facilitates to obtain a globally optimum genetic sequence.

In a particular implementation, the numerical values of the first probability, the second probability and the third probability may be adjusted according to practical demands, which is not limited in the present embodiment.

The task dispatching method according to the present embodiment will be illustrated below.

It is assumed that m=2 and n=10, or, in other words, the quantity of the examining apparatuses is 2 and the quantity of the examination tasks is 10, the task identifications of the 10 examination tasks are 1, 2, . . . , 10, and the examination types of the 10 examination tasks are H, A, T, H, H, A, T, A and T. In the process of the initialization of the generation of the genetic sequences, the 10 examination tasks may be randomly allocated to 2 examining apparatuses, for example, an apparatus 1 and an apparatus 2. Particularly, the task identifications 1-10 of the 10 examination tasks may be randomly encoded to generate the following genetic sequences:
the apparatus 1: \textcircled{6}\textcircled{2}\textcircled{10}\textcircled{7}\textcircled{4}
the apparatus 2: \textcircled{8}\textcircled{9}\textcircled{4}\textcircled{2}\textcircled{5}

The examination order of the 10 examination tasks in the genetic sequences is \textcircled{6}\textcircled{8}\textcircled{2}\textcircled{9}\textcircled{10}\textcircled{3}\textcircled{7}\textcircled{1}\textcircled{4}\textcircled{5}, and according to the examination order of the examination tasks in the genetic sequences, the examination identifications of the examination tasks can be determined. For example, the examination identification in the genetic sequences of the examination task whose task identification is 6 is 1, the examination identification in the genetic sequences of the examination task whose task identification is 8 is 2, and the rest can be done in the same manner.

The operation durations for which the two examining apparatuses completely process the 10 examination tasks according to the above genetic sequences are 30 minutes and 45 minutes, and accordingly the apparatus operation duration corresponding to the genetic sequences is the maximum value of the operation durations of the two examining apparatuses, i.e., 45 minutes.

After the high-quality genetic sequence has been selected, the process of performing cross treatment to two different high-quality genetic sequences may be performed according to the following steps:

It is assumed that the two different high-quality genetic sequences are a parent 1 and a parent 2:
the apparatus 1: \textcircled{6}\textcircled{2}\textcircled{10}\textcircled{7}\textcircled{4} the apparatus 1: \textcircled{3}\textcircled{8}\textcircled{1}\textcircled{7}\textcircled{6}
the apparatus 2: \textcircled{8}\textcircled{9}\textcircled{3}\textcircled{1}\textcircled{5} the apparatus 2: \textcircled{10}\textcircled{2}\textcircled{9}\textcircled{5}\textcircled{4}
the parent 1 the parent 2

Subsequently, several random ones of the genes in the parent 1 and the parent 2 are randomly selected as fixed genes, wherein the fixed genes are expressed by overstriking and italic. The two high-quality genetic sequences of the parent 1 and the parent 2 undergo cross treatment, to generate one offspring, wherein the positions of the fixed genes in the parents are maintained unchanged in the offspring, as shown in the following:
the apparatus 1: ◯\textcircled{2}\textcircled{10}◯◯
the apparatus 2: ◯\textcircled{9}\textcircled{3}◯◯
the offspring

Subsequently, the genes in the parent 2 different from the fixed genes (expressed by underline) are placed into the offspring sequentially according to the order in the parent 2 and a correspondence relation with the examining apparatuses, to obtain the following offspring, i.e., the genetic sequence that has been updated:
the apparatus 1: \textcircled{3}\textcircled{8}\textcircled{1}\textcircled{7}\textcircled{6}→the apparatus 1: \textcircled{8}\textcircled{2}\textcircled{10}\textcircled{1}\textcircled{6}
the apparatus 2: \textcircled{10}\textcircled{2}\textcircled{9}\textcircled{5}\textcircled{4} the apparatus 2: ,crc 9\textcircled{7}\textcircled{3}, \textcircled{5}\textcircled{4}
the parent 2 the offspring

In the task dispatching method according to the present embodiment, because each of the apparatus operation duration and the task-switching parameter of the genetic sequence has a relation of negative correlation with the adaptive value of the genetic sequence, in the process of each time of the iterative updating, the genetic sequence having a lower apparatus operation duration and a lower task-switching parameter has a higher probability of being selected. Accordingly, by performing multiple times of iterative updating to the genetic sequences, the examination order of the examination tasks can be optimized and adjusted. In an aspect, that reaches the objective of shortening the longest apparatus operation duration, whereby the operation durations of the examining apparatuses are more averaged, to prevent the case in which some of the examining apparatuses operate with a high load while the other apparatuses are idle, and, in another aspect, that reaches the objective of shortening the duration of task switching, which can increase the utilization ratio of the apparatuses, and increase the efficiency of examination.

The executive subject of the present embodiment may also be connected to a display. The display may be used to display the information of the examination task that currently prepares to be examined (for example, the task identification of the examination task, the corresponding patient name, and so on) and the information of the examining apparatus undertaking to process the examination task (for example, the room number corresponding to the examining apparatus, and so on), and may also be used to display the information of the examination task that prepares to be examined next, and so on.

The executive subject of the present embodiment may also be connected to a player. The player may be used to perform voice broadcasting with respect to the information of the examination task that currently prepares to be examined and the information of the examining apparatus undertaking to process the examination task.

It should be noted that, assuming that, before the n examination tasks, m examination tasks have already been completely processed or are being processed, and before the processing of the m examination tasks all of the m examining apparatuses are idle, then the m examination tasks may be randomly allocated to the m examining apparatuses. That can increase the utilization ratio of the examining apparatuses, reduce the idleness time of the examining apparatuses, and increase the efficiency of dispatching. The n examination tasks acquired after the m examination tasks may be dispatched by using the task dispatching method according to the present embodiment. Moreover, in order to reduce the time quantity of calculation, the process of the task dispatching with respect to the n examination tasks may be completed before the ending of the examination tasks that are being processed in any one of the examining apparatuses, which cannot only reduce the time quantity of calculation, but also can increase the utilization ratio of the examining apparatuses, reduce the idleness time of the examining apparatuses, and increase the efficiency of dispatching.

After the dispatching scheme of the n examination tasks, i.e., the genetic sequence, has been determined, the examination tasks may be dispatched according to the finally determined genetic sequence. After the examination tasks of a specified quantity have been completely processed according to the finally determined genetic sequence, or after the examination tasks have been processed for a preset duration according to the finally determined genetic sequence, the examination tasks among the n examination tasks that have not started to be processed and the examination tasks acquired after the n examination tasks may be re-determined to be the examination tasks to be dispatched, and then dispatched by using the task dispatching method according to the present embodiment.

In an optional embodiment, in the step S13, the step of determining the apparatus operation duration that corresponds to the genetic sequence before the updating may particularly include:
firstly, acquiring a first historical operation duration of a second examining apparatus, wherein the second examining apparatus refers to any one of the one or more examining apparatuses, and the first historical operation duration refers to a duration during which the second examining apparatus has already started operating before the examination tasks are processed;
subsequently, according to examination durations of predetermined examination types, and an examination type of a second examination task, determining an examination duration of the second examination task, wherein the second examination task refers to an examination task in the genetic sequence before the updating that has a correspondence relation with the second examining apparatus;
subsequently, summing the first historical operation duration with the examination durations of the second examination tasks, to obtain a pre-estimated operation duration of the second examining apparatus; and
subsequently, determining a maximum value of the pre-estimated operation durations of the one or more examining apparatuses to be the apparatus operation duration.

The examination duration of an examination type may be the average value of the durations that are required by one or more examining apparatuses processing the examination type. For example, the examination durations of the three examination types of head magnetic resonance imaging H, thoracic-vertebrae magnetic resonance imaging T and abdomen magnetic resonance imaging A are 10 minutes, 12 minutes and 15 minutes respectively.

The historical operation duration may, for example, be the duration from starting up to operate to the processing of the second examination task by the second examining apparatus within a day.

For example, if the first historical operation duration is 30 minutes, the quantity of the second examination tasks is three, and the examination types are H, A and T, then it can be determined that the examination durations of the three second examination tasks are 10 minutes, 15 minutes and 12 minutes respectively. By summing the first historical operation duration with the examination durations of the second examination tasks, the pre-estimated operation duration of the second examining apparatus is obtained as 10 minutes+15 minutes+12 minutes+30 minutes=67 minutes.

By using the above process, the pre-estimated operation duration of each of the examining apparatuses can be calculated. When the quantity of the examining apparatuses is 2, the pre-estimated operation durations of the two examining apparatuses are 67 minutes and 72 minutes, and accordingly it can be determined that the apparatus operation duration is the maximum value of them, i.e., 72 minutes.

In the present implementation, because the apparatus operation duration includes the historical operation duration, it can be ensured that the total operation durations (for example, within a whole day) of the examining apparatuses are more averaged.

In an optional embodiment, in the step S13, the step of determining the task-switching parameter that corresponds to the genetic sequence before the updating includes: firstly, according to preset similarities between any two examination types, and examination types of the first examination tasks, determining first similarities between any two successive instances of the first examination tasks in the genetic sequence before the updating; subsequently, averaging the first similarities, to obtain a similarity parameter quantity; and subsequently, calculating a reciprocal of the similarity parameter quantity, to obtain the task-switching parameter.

In a particular implementation, the similarity between any two examination types may be preset in the following manner, the similarity between two same examination types is 1, or else is 0. In CT examination, there are plain scan and enhanced scan to the same one site, and the similarity between the two examination types may be defined as 0.5. In a particular implementation, the similarity between any two examination types may be set according to actual situations, which is not limited in the present embodiment.

It is assumed that the genetic sequence before the updating includes five first examination tasks, the examination types are H, A, T, T and A, and any two successive first examination tasks are H and A, A and T, T and T, and T and A. It can be determined that the first similarities between the any two successive first examination tasks are 0, 0, 1 and 0 respectively, by calculating the average value it is obtained that the similarity parameter quantity is 0.25, and in turn the reciprocal of the similarity parameter quantity is solved, to determine that the task-switching parameter is 4.

In the present implementation, determining the task-switching parameter according to the similarity between two successive examination tasks in the same one examining apparatus can increase the efficiency of the task dispatching, and increase the accuracy of the task-switching parameter.

In an optional embodiment, in the step S13, the step of, according to the apparatus operation duration and the task-switching parameter, determining the adaptive value of the genetic sequence before the updating includes: firstly, acquiring a reference operation duration; and subsequently, calculating a difference between the reference operation duration and the apparatus operation duration, and performing weighted summation to the difference and a reciprocal of the task-switching parameter, to obtain the adaptive value.

The reference operation duration may be the maximum operation duration of the examining apparatuses that is determined by using the mode of dispatching of First Come First Serve. It may also be determined according to the apparatus operation durations of the genetic sequences in the previous round of the process of iterative updating. For example, it may be the average value of the apparatus operation durations of a plurality of genetic sequences. The mode of determining the reference operation duration is not limited in the present embodiment.

Particularly, the adaptive value may be calculated by using the following formula:

f(x)=0.6*(C1−C)+0.4*s
wherein f(x) represents the adaptive value of the genetic sequence before the updating x, C1 represents the reference operation duration, C represents the apparatus operation duration, and s represents the reciprocal of the task-switching parameter. It should be noted that, because the task-switching parameter and the similarity parameter quantity are the reciprocals of each other, s may also represent the similarity parameter quantity. The weight coefficient of the difference may be greater than the weight coefficient of the reciprocal of the task-switching parameter. In the above formula, the weight coefficient of the difference is 0.6, and the weight coefficient of the reciprocal of the task-switching parameter is 0.4. In a particular implementation, the weight coefficients may be adjusted according to actual situations, which is not limited in the present embodiment.

When the reference operation duration is the maximum operation duration of the examining apparatuses that is determined by using the mode of dispatching of First Come First Serve, the step of acquiring the reference operation duration may particularly include: firstly, acquiring a second historical operation duration of a third examining apparatus, wherein the third examining apparatus refers to any one of the one or more examining apparatuses, and the second historical operation duration refers to a duration during which the third examining apparatus has already started operating before the examination tasks are processed; subsequently, according to examination durations of predetermined examination types, the examination types of the examination tasks and an order of acquiring the examination tasks, determining a third examination task required to be processed by the third examining apparatus and an examination duration of the third examination task; subsequently, summing the examination durations of the third examination tasks and the second historical operation duration, to obtain a sequential operation duration of the third examining apparatus; and subsequently, determining a maximum value of the sequential operation durations of the one or more examining apparatuses to be the reference operation duration.

The second historical operation duration refers to the duration during which the third examining apparatus has already started operating before the third examination task is processed.

It is assumed that the second historical operation duration is 45 minutes, the quantity of the third examination tasks allocated to the third examining apparatus in the mode of dispatching of First Come First Serve is three, and the examination types are H, A and T. It can be determined that the examination durations of the three third examination tasks are 10 minutes, 15 minutes and 12 minutes respectively. By summing the examination durations of the third examination tasks and the second historical operation duration, the result is 10 minutes+12 minutes+15 minutes+45 minutes=82 minutes; in other words, the sequential operation duration of the third examining apparatus is 82 minutes.

By using the above process, the sequential operation duration of each of the examining apparatuses can be calculated. When the quantity of the examining apparatuses is 2, the sequential operation durations of the two examining apparatuses are 82 minutes and 95 minutes, and accordingly it can be determined that the reference operation duration is 95 minutes.

In an optional embodiment, in the step S13, a set formed by all of the genetic sequences before the updating is a population, and the step of, according to the adaptive value, selecting the high-quality genetic sequence from the plurality of genetic sequences before the updating includes: firstly, calculating a sum of adaptive values of all of the genetic sequences in the population, to obtain a population adaptive value; subsequently, calculating a ratio of an adaptive value of a first genetic sequence to the population adaptive value, wherein the first genetic sequence refers to any one of all of the genetic sequences before the updating; and subsequently, by using a roulette wheel selection method, by using the ratio as a probability of being-selected, selecting a genetic sequence from the population, wherein the selected genetic sequence is the high-quality genetic sequence.

In a particular implementation, Σf(x) may represent the sum of the adaptive values of all of the genetic sequences in the population, i.e., the population adaptive value, f(x) may represent the adaptive value of any one genetic sequence (i.e., the first genetic sequence) x in the population, and the ratio f(x)/Σf(x) of the adaptive value of the any one genetic sequence x to the population adaptive value may represent the capacity of the first genetic sequence x of generating an offspring. By using a roulette wheel selection method, this step may include, by using the ratios corresponding to the genetic sequences in the population as the selection probabilities, selecting a genetic sequence from the population, to obtain the high-quality genetic sequence.

In the present implementation, before the step of calculating the sum of adaptive values of all of the genetic sequences in the population, the method may further include the following steps: if an adaptive value of a second genetic sequence is less than a first preset threshold, deleting the second genetic sequence from the population; and if the adaptive value of the second genetic sequence is equal to the first preset threshold, adjusting the adaptive value of the second genetic sequence to be a sum of the first preset threshold and a specified numerical value.

The second genetic sequence refers to any one of all of the genetic sequences before the updating. The first preset threshold is greater than or equal to 0. The specified numerical value is greater than 0 and less than or equal to 0.1. For example, the first preset threshold may be 0. When the first preset threshold is 0, the specified numerical value may be a very small positive number, for example, 0.05.

By deleting the second genetic sequence whose adaptive value is less than the first preset threshold, it can be ensured that the apparatus operation durations corresponding to the genetic sequences in the population are less than the reference operation duration.

By adjusting the adaptive value equal to the first preset threshold to be the sum between the first preset threshold and the specified numerical value, error reporting in the calculating process can be prevented.

In an optional embodiment, when the genetic sequence that has been updated is a plurality of genetic sequences that have been updated, in the step S13, the step of, according to the genetic sequence that has been updated, dispatching the examination tasks includes: firstly, calculating adaptive values of the genetic sequences that have been updated; subsequently, selecting a genetic sequence whose adaptive value is greater than or equal to a second preset threshold as a target genetic sequence from the plurality of genetic sequences that have been updated; and subsequently, according to the target genetic sequence, dispatching the examination tasks.

In a particular implementation, the mode of calculating the adaptive values of the genetic sequences that have been updated may be the same as the mode of calculating the adaptive values of the genetic sequences before the updating, and is not discussed here further.

The second preset threshold may be the maximum value of the adaptive values of the plurality of genetic sequences that have been updated, and may also be set according to practical demands, which is not limited in the present embodiment.

For example, when the quantity of the genetic sequences that have been updated is two, the adaptive value of the two genetic sequences that have been updated are 5 and 7. Accordingly, the genetic sequence of the adaptive value of 7 may be determined as the target genetic sequence, and in turn the examination tasks are dispatched according to the target genetic sequence.

In an optional embodiment, the task identifications are identifications that are determined according to the order of acquiring the examination tasks, and in the step S13, before the step of determining the apparatus operation duration and the task-switching parameter that correspond to the genetic sequence before the updating, the method may further include: firstly, according to an examination order of the examination tasks in a third genetic sequence, determining examination identifications of the examination tasks, wherein the third genetic sequence refers to any one of all of the genetic sequences before the updating; subsequently, calculating differences between the examination identifications and the task identifications; and if any one of the differences is less than a third preset threshold, deleting the third genetic sequence.

The third preset threshold may, for example, be −5, which may be particularly determined according to practical demands, and is not limited in the present embodiment.

Particularly, this step may include firstly according to an examination order of the examination tasks in a third genetic sequence, determining examination identifications of the examination tasks, subsequently calculating differences between the examination identifications and the task identifications, and if the third genetic sequence has a difference less than the third preset threshold, deleting the third genetic sequence from the population formed by the genetic sequences before the updating.

The present implementation can ensure that a firstly arriving patient is not arranged too late in the examination order, and take into consideration the case of postponing in the examination order while optimizing the apparatus operation durations, to enable the determined task dispatching theme to be more rationalized and humanized.

FIG. 2 is a block diagram of the task dispatching apparatus according to an illustrative embodiment. Referring to FIG. 2, the apparatus may include:
an acquiring module 21 configured for acquiring a plurality of examination tasks to be dispatched and one or more examining apparatuses configured for processing the plurality of examination tasks, wherein the examination tasks have different task identifications;
a generating module 22 configured for, according to the examination tasks and the examining apparatus, generating a genetic sequence, the genetic sequence includes a correspondence relation between the examination tasks and the examining apparatuses processing the examination tasks, and genes in the genetic sequence are the task identifications of the examination tasks; and
an updating module 23 configured for performing iterative updating to the genetic sequence, till an iteration stopping condition is satisfied, and according to the genetic sequence that has been updated, dispatching the examination tasks;
wherein the updating module 23 is particularly configured for:
determining an apparatus operation duration and a task-switching parameter that correspond to the genetic sequence before the updating, wherein the apparatus operation duration refers to a maximum value of operation durations during which the one or more examining apparatuses completely process the plurality of examination tasks according to the genetic sequence before the updating, the task-switching parameter is for characterizing a duration that is required by a first examining apparatus corresponding to the maximum value for switching and processing a first examination task, and the first examination task refers to an examination task in the genetic sequence before the updating that has a correspondence relation with the first examining apparatus;
according to the apparatus operation duration and the task-switching parameter, determining an adaptive value of the genetic sequence before the updating, wherein each of the apparatus operation duration and the task-switching parameter has a relation of negative correlation with the adaptive value; and
according to the adaptive value, selecting a high-quality genetic sequence from a plurality of genetic sequences before the updating, and generating the genetic sequence that has been updated based on the high-quality genetic sequence.

The particular modes of the operations performed by the modules of the apparatus according to the above embodiment have already been described in detail in the embodiments of the method, for example, implemented in the form of software, hardware, firmware and so on, and will not be explained and described in detail herein.

The above-described device embodiments are merely illustrative, wherein the units that are described as separate components may or may not be physically separate, and the components that are displayed as units may or may not be physical units; in other words, they may be located at the same one location, and may also be distributed to a plurality of network units. Some or all of the modules may be selected according to the actual demands to realize the purposes of the solutions of the embodiments. A person skilled in the art can understand and implement the technical solutions without paying creative work.

Each component embodiment of the present disclosure may be implemented by hardware, or by software modules that are operated on one or more processors, or by a combination thereof. A person skilled in the art should understand that some or all of the functions of some or all of the components of the computing and processing device according to the embodiments of the present disclosure may be implemented by using a microprocessor or a digital signal processor (DSP) in practice. The present disclosure may also be implemented as apparatus or device programs (for example, computer programs and computer program products) for implementing part of or the whole of the method described herein. Such programs for implementing the present disclosure may be stored in a computer-readable medium, or may be in the form of one or more signals. Such signals may be downloaded from an Internet website, or provided on a carrier signal, or provided in any other forms.

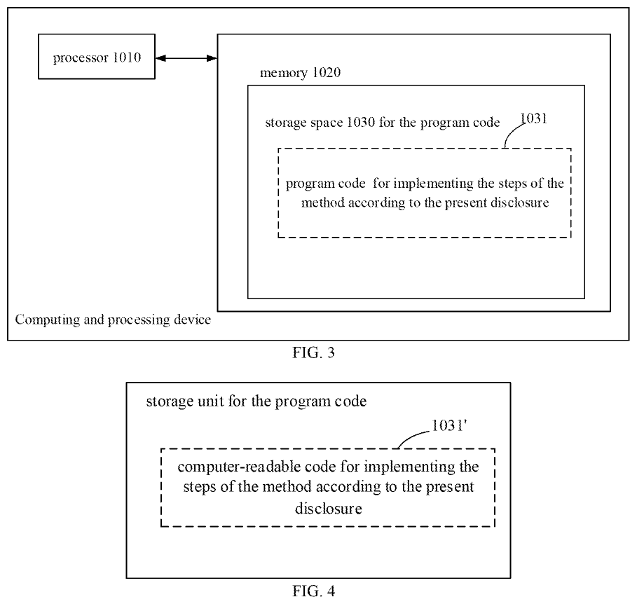

For example, FIG. 3 shows a computing and processing device that can implement the method according to the present disclosure. The computing and processing device traditionally include a processor 1010 and a computer program product or computer-readable medium in the form of a memory 1020. The memory 1020 may be electronic memories such as flash memory, EEPROM (Electrically Erasable Programmable Read Only Memory), EPROM, hard disk or ROM. The memory 1020 has the storage space 1030 of the program code 1031 for implementing any steps of the above method. For example, the storage space 1030 for program code may contain program codes 1031 for individually implementing each of the steps of the above method. Those program codes may be read from one or more computer program products or be written into the one or more computer program products. Those computer program products include program code carriers such as a hard disk, a compact disk (CD), a memory card or a floppy disk. Such computer program products are usually portable or fixed storage units as shown in FIG. 4. The storage unit may have storage segments or storage spaces with similar arrangement to the memory 1020 of the computing and processing device in FIG. 3. The program codes may, for example, be compressed in a suitable form. Generally, the storage unit contains a computer-readable code 1031′, which can be read by a processor like 1010. When those codes are executed by the computing and processing device, the codes cause the computing and processing device to implement each of the steps of the method described above.

The embodiments of the description are described in the mode of progression, each of the embodiments emphatically describes the differences from the other embodiments, and the same or similar parts of the embodiments may refer to each other.

Finally, it should also be noted that, in the present text, relation terms such as first and second are merely intended to distinguish one entity or operation from another entity or operation, and that does not necessarily require or imply that those entities or operations have therebetween any such actual relation or order. Furthermore, the terms “include”, “include” or any variants thereof are intended to cover non-exclusive inclusions, so that processes, methods, articles or devices that include a series of elements do not only include those elements, but also include other elements that are not explicitly listed, or include the elements that are inherent to such processes, methods, articles or devices. Unless further limitation is set forth, an element defined by the wording “including a . . . ” does not exclude additional same element in the process, method, article or device including the element.

The task dispatching method and apparatus, the computing and processing device, the computer program and the computer-readable medium according to the present disclosure have been described in detail above. The principle and the embodiments of the present disclosure are described herein with reference to the particular examples, and the description of the above embodiments is merely intended to facilitate to understand the method according to the present disclosure and its core concept. Moreover, for a person skilled in the art, according to the concept of the present disclosure, the particular embodiments and the range of application may be varied. In conclusion, the contents of the description should not be understood as limiting the present disclosure.

It should be understood that, although the steps in the flow charts in the drawings are shown sequentially according to the indication by the arrows, those steps are not necessarily performed sequentially according to the sequence indicated by the arrows. Unless expressly described herein, the sequence of the performances of those steps are not strictly limited, and they may be performed in other sequences. Furthermore, at least some of the steps in the flow charts in the drawings may include a plurality of sub-steps or a plurality of stages, wherein those sub-steps or stages are not necessarily completely performed at the same one moment, but may be performed at different moments, and their performance sequence is not necessarily sequential performance, but may be performance alternate with at least some of the other steps or the sub-steps or stages of the other steps.

A person skilled in the art, after considering the description and implementing the invention disclosed herein, will readily envisage other embodiments of the present disclosure. The present disclosure aims at encompassing any variations, uses or adaptative alternations of the present disclosure, wherein those variations, uses or adaptative alternations follow the general principle of the present disclosure and include common knowledge or common technical means in the art that are not disclosed by the present disclosure. The description and the embodiments are merely deemed as exemplary, and the true scope and spirit of the present disclosure are presented by the following claims.

It should be understood that the present disclosure is not limited to the accurate structure that has been described above and shown in the drawings, and may have various modifications and variations without departing from its scope. The scope of the present disclosure is merely limited by the appended claims.

The “one embodiment”, “an embodiment” or “one or more embodiments” as used herein means that particular features, structures or characteristics described with reference to an embodiment are included in at least one embodiment of the present disclosure. Moreover, it should be noted that here an example using the wording “in an embodiment” does not necessarily refer to the same one embodiment.

The description provided herein describes many concrete details. However, it can be understood that the embodiments of the present disclosure may be implemented without those concrete details. In some of the embodiments, well-known processes, structures and techniques are not described in detail, so as not to affect the understanding of the description.

In the claims, any reference signs between parentheses should not be construed as limiting the claims. The word “include” does not exclude elements or steps that are not listed in the claims. The word “a” or “an” preceding an element does not exclude the existing of a plurality of such elements. The present disclosure may be implemented by means of hardware including several different elements and by means of a properly programmed computer. In unit claims that list several devices, some of those devices may be embodied by the same item of hardware. The words first, second, third and so on do not denote any order. Those words may be interpreted as names.

Finally, it should be noted that the above embodiments are merely intended to explain the technical solutions of the present disclosure, and not to limit them. Although the present disclosure is explained in detail with reference to the above embodiments, a person skilled in the art should understand that he can still modify the technical solutions set forth by the above embodiments, or make equivalent substitutions to part of the technical features of them. However, those modifications or substitutions do not make the essence of the corresponding technical solutions depart from the spirit and scope of the technical solutions of the embodiments of the present disclosure.

1. A task dispatching method, wherein the task dispatching method comprises:
acquiring a plurality of examination tasks to be dispatched and one or more examining apparatuses configured for processing the plurality of examination tasks, wherein the examination tasks have different task identifiers;
according to the examination tasks and the examining apparatus, generating a genetic sequence, the genetic sequence comprises a correspondence relation between the examination tasks and the examining apparatuses processing the examination tasks, and genes in the genetic sequence are the task identifiers of the examination tasks; and
performing iterative updating to the genetic sequence, till an iteration stopping condition is satisfied, and according to the genetic sequence that has been updated, dispatching the examination tasks;
wherein the step of the iterative updating comprises:
determining an apparatus operation duration and a task-switching parameter that correspond to the genetic sequence before the updating, wherein the apparatus operation duration refers to a maximum value of operation durations during which the one or more examining apparatuses completely process the plurality of examination tasks by using the genetic sequence before the updating, the task-switching parameter is for characterizing a duration that is required by a first examining apparatus corresponding to the maximum value for switching and processing a first examination task, and the first examination task refers to an examination task in the genetic sequence before the updating that has a correspondence relation with the first examining apparatus;
according to the apparatus operation duration and the task-switching parameter, determining an adaptive value of the genetic sequence before the updating, wherein each of the apparatus operation duration and the task-switching parameter has a relation of negative correlation with the adaptive value; and
according to the adaptive value, selecting a high-quality genetic sequence from a plurality of genetic sequences before the updating, and generating the genetic sequence that has been updated based on the high-quality genetic sequence.

2. The task dispatching method according to claim 1, wherein the step of determining the apparatus operation duration that corresponds to the genetic sequence before the updating comprises:
acquiring a first historical operation duration of a second examining apparatus, wherein the second examining apparatus refers to any one of the one or more examining apparatuses, and the first historical operation duration refers to a duration during which the second examining apparatus has already started operating before the examination tasks are processed;
according to examination durations of predetermined examination types, and an examination type of a second examination task, determining an examination duration of the second examination task, wherein the second examination task refers to an examination task in the genetic sequence before the updating that has a correspondence relation with the second examining apparatus;
summing the first historical operation duration with the examination durations of the second examination tasks, to obtain a pre-estimated operation duration of the second examining apparatus; and
determining a maximum value of the pre-estimated operation durations of the one or more examining apparatuses to be the apparatus operation duration.

3. The task dispatching method according to claim 1, wherein the step of determining the task-switching parameter that corresponds to the genetic sequence before the updating comprises:
according to preset similarities between any two examination types, and examination types of the first examination tasks, determining first similarities between any two successive instances of the first examination tasks in the genetic sequence before the updating;
averaging the first similarities, to obtain a similarity parameter quantity; and
calculating a reciprocal of the similarity parameter quantity, to obtain the task-switching parameter.

4. The task dispatching method according to claim 1, wherein the step of, according to the apparatus operation duration and the task-switching parameter, determining the adaptive value of the genetic sequence before the updating comprises:
acquiring a reference operation duration; and
calculating a difference between the reference operation duration and the apparatus operation duration, and performing weighted summation to the difference and a reciprocal of the task-switching parameter, to obtain the adaptive value.

5. The task dispatching method according to claim 4, wherein the step of acquiring the reference operation duration comprises:
acquiring a second historical operation duration of a third examining apparatus, wherein the third examining apparatus refers to any one of the one or more examining apparatuses, and the second historical operation duration refers to a duration during which the third examining apparatus has already started operating before the examination tasks are processed;
according to examination durations of predetermined examination types, the examination types of the examination tasks and an order of acquiring the examination tasks, determining a third examination task required to be processed by the third examining apparatus and an examination duration of the third examination task;
summing the examination durations of the third examination tasks and the second historical operation duration, to obtain a sequential operation duration of the third examining apparatus; and
determining a maximum value of the sequential operation durations of the one or more examining apparatuses to be the reference operation duration.

6. The task dispatching method according to claim 1, wherein a set formed by all of the genetic sequences before the updating is a population, and the step of, according to the adaptive value, selecting the high-quality genetic sequence from the plurality of genetic sequences before the updating comprises:
calculating a sum of adaptive values of all of the genetic sequences in the population, to obtain a population adaptive value;
calculating a ratio of an adaptive value of a first genetic sequence to the population adaptive value, wherein the first genetic sequence refers to any one of all of the genetic sequences before the updating; and
by using a roulette wheel selection method, by using the ratio as a probability of being-selected, selecting a genetic sequence from the population, wherein the selected genetic sequence is the high-quality genetic sequence.

7. The task dispatching method according to claim 6, wherein before the step of calculating the sum of adaptive values of all of the genetic sequences in the population, the method further comprises:
if an adaptive value of a second genetic sequence is less than a first preset threshold, deleting the second genetic sequence from the population; and
if the adaptive value of the second genetic sequence is equal to the first preset threshold, adjusting the adaptive value of the second genetic sequence to be a sum between the first preset threshold and a specified numerical value;
wherein the second genetic sequence refers to any one of all of the genetic sequences before the updating;
the first preset threshold is greater than or equal to 0; and
the specified numerical value is greater than 0 and less than or equal to 0.1.

8. The task dispatching method according to claim 1, wherein the step of generating the genetic sequence that has been updated based on the high-quality genetic sequence comprises at least one of the following steps:
according to a first probability, replicating the high-quality genetic sequence, to generate the genetic sequence that has been updated;
according to a second probability, performing cross treatment to two different instances of the high-quality genetic sequence, to generate the genetic sequence that has been updated; and
according to a third probability, performing variation treatment to genetic sequences obtained after cross treatment, to generate the genetic sequence that has been updated.

9. The task dispatching method according to claim 1, wherein the step of, according to the examination tasks and the examining apparatus, generating the genetic sequence comprises:
allocating the plurality of examination tasks randomly to the one or more examining apparatuses, and according to a correspondence relation between the allocated examination tasks and the examining apparatuses, determining the genetic sequence.

10. The task dispatching method according to claim 1, wherein the genetic sequence that has been updated is a plurality of genetic sequences that have been updated, and the step of, according to the genetic sequence that has been updated, dispatching the examination tasks comprises:
calculating adaptive values of the genetic sequences that have been updated;
selecting from the plurality of genetic sequences that have been updated a genetic sequence whose adaptive value is greater than or equal to a second preset threshold as a target genetic sequence; and
according to the target genetic sequence, dispatching the examination tasks.

11. The task dispatching method according to claim 1, wherein the task identifiers are identifiers that are determined according to the order of acquiring the examination tasks, and before the step of determining the apparatus operation duration and the task-switching parameter that correspond to the genetic sequence before the updating, the method further comprises:
according to an examination order of the examination tasks in a third genetic sequence, determining examination identifiers of the examination tasks, wherein the third genetic sequence refers to any one of all of the genetic sequences before the updating;
calculating differences between the examination identifiers and the task identifiers; and
if any one of the differences is less than a third preset threshold, deleting the third genetic sequence.

12. A task dispatching apparatus, wherein the task dispatching apparatus comprises:
an acquiring module configured for acquiring a plurality of examination tasks to be dispatched and one or more examining apparatuses configured for processing the plurality of examination tasks, wherein the examination tasks have different task identifiers;
a generating module configured for, according to the examination tasks and the examining apparatus, generating a genetic sequence, the genetic sequence comprises a correspondence relation between the examination tasks and the examining apparatuses processing the examination tasks, and genes in the genetic sequence are the task identifiers of the examination tasks; and
an updating module configured for performing iterative updating to the genetic sequence, till an iteration stopping condition is satisfied, and according to the genetic sequence that has been updated, dispatching the examination tasks;
wherein the updating module is particularly configured for;
determining an apparatus operation duration and a task-switching parameter that correspond to the genetic sequence before the updating, wherein the apparatus operation duration refers to a maximum value of operation durations during which the one or more examining apparatuses completely process the plurality of examination tasks by using the genetic sequence before the updating, the task-switching parameter is for characterizing a duration that is required by a first examining apparatus corresponding to the maximum value for switching and processing a first examination task, and the first examination task refers to an examination task in the genetic sequence before the updating that has a correspondence relation with the first examining apparatus;
according to the apparatus operation duration and the task-switching parameter, determining—an adaptive value of the genetic sequence before the updating, wherein each of the apparatus operation duration and the task-switching parameter has a relation of negative correlation with the adaptive value; and
according to the adaptive value, selecting a high-quality genetic sequence from a plurality of genetic sequences before the updating, and generating the genetic sequence that has been updated based on the high-quality genetic sequence.

13. A calculating and processing device, wherein the calculating and processing device comprises:
a memory storing a computer-readable code; and
one or more processors, wherein when the computer-readable code is executed by the one or more processors, the calculating and processing device implements the task dispatching method according to claim 1.

14. (canceled)

15. A computer-readable medium, wherein the computer-readable medium stores the task dispatching method according to claim 1.

16. The task dispatching method according to claim 2, wherein the task identifiers are identifiers that are determined according to the order of acquiring the examination tasks, and before the step of determining the apparatus operation duration and the task-switching parameter that correspond to the genetic sequence before the updating, the method further comprises:
according to an examination order of the examination tasks in a third genetic sequence, determining examination identifiers of the examination tasks, wherein the third genetic sequence refers to any one of all of the genetic sequences before the updating;
calculating differences between the examination identifiers and the task identifiers; and
if any one of the differences is less than a third preset threshold, deleting the third genetic sequence.

17. The task dispatching method according to claim 3, wherein the task identifiers are identifiers that are determined according to the order of acquiring the examination tasks, and before the step of determining the apparatus operation duration and the task-switching parameter that correspond to the genetic sequence before the updating, the method further comprises:
according to an examination order of the examination tasks in a third genetic sequence, determining examination identifiers of the examination tasks, wherein the third genetic sequence refers to any one of all of the genetic sequences before the updating;
calculating differences between the examination identifiers and the task identifiers; and
if any one of the differences is less than a third preset threshold, deleting the third genetic sequence.

18. The task dispatching method according to claim 4, wherein the task identifiers are identifiers that are determined according to the order of acquiring the examination tasks, and before the step of determining the apparatus operation duration and the task-switching parameter that correspond to the genetic sequence before the updating, the method further comprises:
according to an examination order of the examination tasks in a third genetic sequence, determining examination identifiers of the examination tasks, wherein the third genetic sequence refers to any one of all of the genetic sequences before the updating;
calculating differences between the examination identifiers and the task identifiers; and
if any one of the differences is less than a third preset threshold, deleting the third genetic sequence.

19. The task dispatching method according to claim 5, wherein the task identifiers are identifiers that are determined according to the order of acquiring the examination tasks, and before the step of determining the apparatus operation duration and the task-switching parameter that correspond to the genetic sequence before the updating, the method further comprises:
according to an examination order of the examination tasks in a third genetic sequence, determining examination identifiers of the examination tasks, wherein the third genetic sequence refers to any one of all of the genetic sequences before the updating;
calculating differences between the examination identifiers and the task identifiers; and
if any one of the differences is less than a third preset threshold, deleting the third genetic sequence.

20. The task dispatching method according to claim 6, wherein the task identifiers are identifiers that are determined according to the order of acquiring the examination tasks, and before the step of determining the apparatus operation duration and the task-switching parameter that correspond to the genetic sequence before the updating, the method further comprises:
according to an examination order of the examination tasks in a third genetic sequence, determining examination identifiers of the examination tasks, wherein the third genetic sequence refers to any one of all of the genetic sequences before the updating;
calculating differences between the examination identifiers and the task identifiers; and
if any one of the differences is less than a third preset threshold, deleting the third genetic sequence.

21. The task dispatching method according to claim 7, wherein the task identifiers are identifiers that are determined according to the order of acquiring the examination tasks, and before the step of determining the apparatus operation duration and the task-switching parameter that correspond to the genetic sequence before the updating, the method further comprises:
according to an examination order of the examination tasks in a third genetic sequence, determining examination identifiers of the examination tasks, wherein the third genetic sequence refers to any one of all of the genetic sequences before the updating;
calculating differences between the examination identifiers and the task identifiers; and
if any one of the differences is less than a third preset threshold, deleting the third genetic sequence.
